(19) United States
(12) Patent Application Publication
(10) Pub. No.: US 2024/0029835 A1
(43) Pub. Date: Jan. 25, 2024

(54) Process for Predictive Optimization Algorithm Development for Molecular Structural Synthesis

(71) Applicant: Anika Mahajan Jena, San Ramon, CA (US)

(72) Inventor: Anika Mahajan Jena, San Ramon, CA (US)

(21) Appl. No.: 18/468,731

(22) Filed: Sep. 17, 2023

(51) Int. Cl. G16C 20/50; G16C 20/20

(52) U.S. Cl. CPC G16C 20/50; G16C 20/20

(57) ABSTRACT

The invention provides a process for developing a quantitative algorithm to illustrate and predict the outcome of molecular structural synthesis, from optimized component formulations, for result optimization. Formulations, such as those involved in molecular structural synthesis, involve multiple interacting components. Performance lies on a gradient success scale. Iterative formulations, syntheses, and evaluation under controlled fabrication parameters are followed by rounds of regression and integration. The result of integration is a predictive algorithm that contains all salient component quantities as variables and presents an optimized formulation and risk-tolerance region. The algorithm's output formulations yield optimally performing, efficient syntheses under the specified conditions desired.

### BACKGROUND ON THE INVENTION

Molecular structures are synthesized from component formulations. Synthesizers have historically been reliant on long-term evolutionary development using personal recollection of qualitative observations from past syntheses. They consider several contributing factors and a gradient measure of success. They must recall synthesis results from months or years prior. Unfortunately, qualitatively founded formulations most often yield inadequate products that deviate from the desired result. This is especially concerning because molecular structure synthesis is often time- and resource-intensive.

### SUMMARY OF THE INVENTION

The present invention provides a quantitatively based process for risk-informed optimized molecular formulation development using iterative formulation, synthesis, and evaluation under controlled fabrication parameters. The invention comprises the following steps:
a. Identification of formulation components salient to process performance, potentially with all but one component contributing fundamentally to the characteristics of the desired product, and one component being more manipulable;
b. Definition of process performance value on a fixed numerical scale 0 to n;
c. Listing of iterative formulation sets, each set containing formulations defining quantities of all salient components, and all component quantities held constant within a set except for a single incrementally altered target component;
d. Synthesis of each listed formulation;
e. Result observation and performance evaluation, with record of each formulation and its performance value;
f. At least one round of mathematical regression of each formulation set against its results, yielding equations modeling the effect of each component on the system under a specific component condition set;
g. At least one round of result integration.

The process yields an algorithm involving performance value and all salient components. This algorithm may be applied in subsequent formulation development. For example, the algorithm may take, as input, all desired component quantities except for a manipulable component. The algorithm would then return a distribution curve of performance values versus manipulable component quantity. The domain of optimal performance includes the formulation for highest performance value, as well as regions of slight risk. Therefore, the optimal formulation may be selected from this region considering user risk-tolerance.

### BRIEF DESCRIPTION OF THE FIGURES

FIG. 1 depicts a partial possibility of formulation sets involving one incremented target component (labeled D) and other components (labeled A, B, and C) strategically altered in anticipation of variable-based regressions.

FIG. 2 flowcharts one round of regression used to target a component variable, and suggests the mathematical process mimicked by the regression.

FIG. 3 illustrates component dependency through a plot illustrating several conditions of one component variable and their effect on another component variable. Each quantity of variable B results in different behavior for variable D, mathematically intertwining the variables and introducing necessity for regression and integration.

FIG. 4 displays a sample result of the developed algorithm, in which specified desirable component quantities are embedded, and an optimized manipulable component quantity may be selected from the risk-tolerance region.

### DETAILED DESCRIPTION OF THE INVENTION

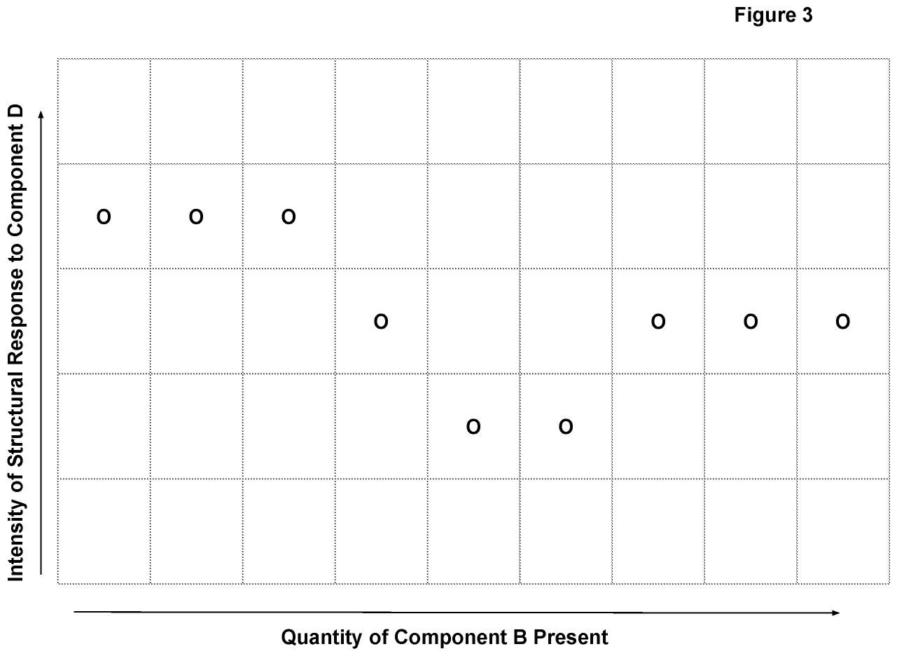

The inventor has developed a process for developing an algorithm for predictive outcome illustration of molecular formulations to achieve optimized synthesis. This applies primarily to molecular structure synthesis and uniquely considers cases such as FIG. 3, in which altering the quantity of one structural component changes the effect of another structural component on characteristics or success of the product. This dependent variability is often overlooked as stochasticity, but by the present invention, may be experimentally and mathematically observed, analyzed, and accounted for.

The invention comprises the following steps:
a. Identification of formulation components salient to process performance, potentially with all but one component contributing fundamentally to the characteristics of the desired product, and one component being more manipulable.
   Consider a hypothetical structure to be synthesized from six components. Of these six components, two are inert: altering their quantity in the initial formulation has no effect on the likelihood, quality, or extent of product synthesis success. Disregard these components for algorithm development. Four components are salient: altering their quantity significantly affects the likelihood, quality, or extent of product synthesis success. Note these four components as A, B, C, and D. In addition to their effect on performance, hypothetical components A, B, and C affect several desirable aspects of the product, including but not limited to opacity, substructure density, and rigidity. D has a more drastic effect on performance than on desired characteristics. Therefore, it is considered the more manipulable component.
b. Definition of process performance value on a fixed numerical scale from 0 to a positive real value.

In a hypothetical scenario, a synthesis product is required to display a characteristic under a certain number n of conditions and display a product density p. These multiple requirements are consolidated as into a single maximum value integrating both values such as n*p.
c. Listing of iterative formulation sets, each set containing formulations defining quantities of all salient components, and all component quantities held constant within a set except for a single incrementally altered target component;

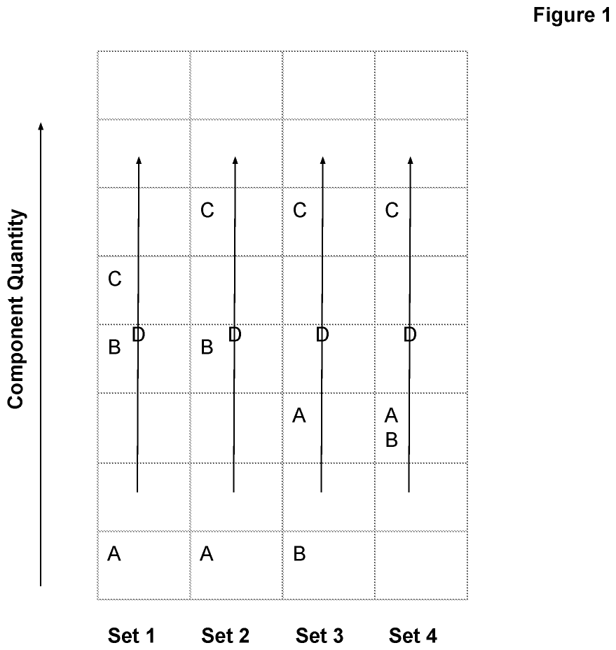

Formulation sets may be developed as in FIG. 1. In the hypothetical formulations presented in the figure, component D is altered in each formulation set. Multiple sets are created to observe the behavior of D under different component conditions. This is necessary to account for the component-dependent variability described in FIG. 3 and previously mentioned.
d. Synthesis of each listed formulation.

It is beneficial to perform all the syntheses formulated in step c in a short time frame for maximum control and uniformity of outside factors, which in a chemical context include but are not limited to water conductivity and equipment status. (Additionally, all later syntheses could utilize the algorithm developed by the process described by the present invention, saving time and resources.)
e. Result observation and performance evaluation, with record of each formulation and its performance value;

The record of each synthesis must include all component quantities that were used for that synthesis. It also must include the assigned performance value P on the objective scale defined in step b, 0<P<max. For ease of step f, records may be organized by set.
f. At least one round of mathematical regression of each set against its results, yielding equations modeling the effect of each component on the system under a specific component condition set.

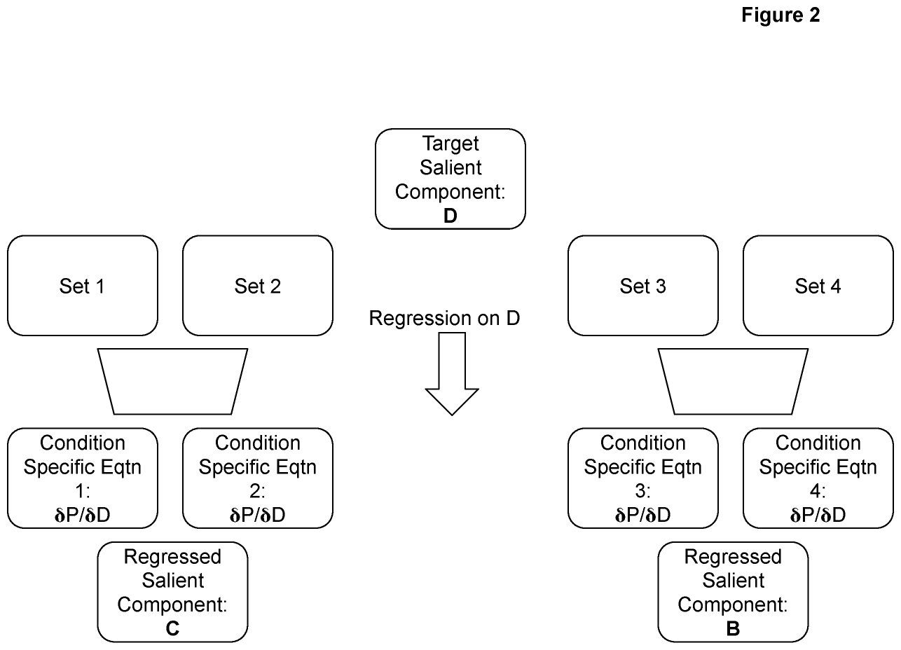

In the hypothetical scenario with salient components A, B, C, and D, and manipulable component D, regressions would yield dP/dD=[equation] for each condition set of fixed A, B, and C by the method described in FIG. 2. Another round of regression—
1. Condensing conditional partials in which only A changes,
2. Condensing conditional partials in which only B changes, and
3. Condensing conditional partials in which only C changes—
   yields (dP/dD)(dD/dA), (dP/dD)(dD/dB), and (dP/dD)(dD/dC), respectively.
g. At least one round of result integration.

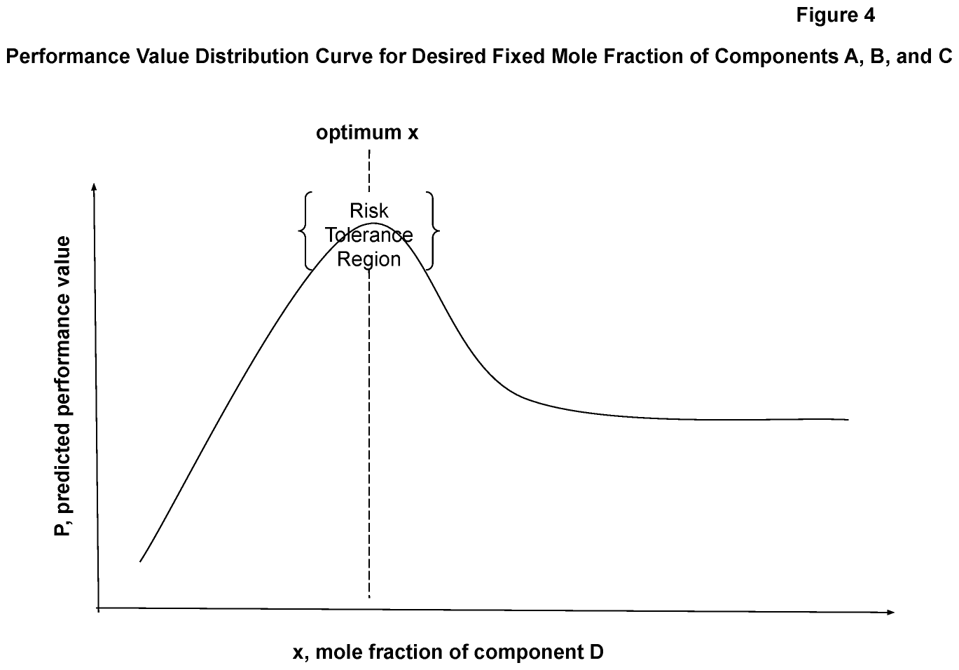

The result of the integration is a predictive algorithm that contains all salient component quantities as variables. If all component variables are fixed by user specification, it will return a predictive P value. If one (or potentially more) variables are left manipulable, it will return a distribution curve of predicted performance values versus manipulable component quantity or quantities, as in FIG. 4. An acceptable risk-tolerance region on the curve may be defined by the user, giving them one suggested optimal formulation and a “grace region” for experimental error or fabrication preference.

1. A process for developing formulations of multiple interacting components, as may be applied to molecular structural synthesis, by a predictive algorithm developed by the following steps,
a. Identification of salient components, potentially with all but one component contributing fundamentally to the characteristics of the desired product, and one component being manipulable;
b. Iterative formulation, synthesis, and objective evaluation of sets, each set containing formulations defining quantities of all salient components, and all component quantities held constant within a set except for a single incrementally altered target component;
c. At least one round of mathematical regression of each formulation set against its results, yielding equations modeling the effect of each component on the system under a specific component condition set;
d. At least one round of result integration.
